(19) United States
(12) Patent Application Publication
(10) Pub. No.: US 2022/0000565 A1
(43) Pub. Date: Jan. 6, 2022

(54) SYSTEM FOR RENAL PUNCTURING ASSISTANCE

(71) Applicants: K.B. Gururaj, Ranebennu (IN); Satish Kalme, Bangalore (IN); S.K. Raghunath, Bangalore (IN); Rakesh Sharma, Hisar (IN)

(72) Inventors: K.B. Gururaj, Ranebennu (IN); Satish Kalme, Bangalore (IN); S.K. Raghunath, Bangalore (IN); Rakesh Sharma, Hisar (IN)

(73) Assignee: Comofi Medtech Private Limited, Bangalore (IN)

(21) Appl. No.: 17/294,009

(22) PCT Filed: Nov. 12, 2019
(86) PCT No.: PCT/IB2019/059685

§ 371 Date: May 14, 2021

(30) Foreign Application Priority Data
Nov. 15, 2018 (IN) IN201841043103A

(51) Int. Cl. A61B 34/20; A61B 34/30; A61B 17/34; A61B 90/00

(52) U.S. Cl. CPC A61B 34/20; A61B 34/30; A61B 17/34; A61B 90/37; A61B 2090/3983; A61B 2034/2065; A61B 2090/365; A61B 2090/3762; A61B 2090/3908; A61B 90/39

(57) ABSTRACT

The present invention relates to a system for renal puncturing assistance in percutaneous nephrolithotomy (PCNL) surgery. The system includes a robotic device having an end effect or including an image acquisition unit, needle holding mechanism and a guide wire mechanism. The needle holding mechanism accommodates a puncture needle with corresponding guide wire provided by the guide wire mechanism. The system includes a computing unit operatively coupled to the image acquisition unit. The system has an AR engine that augments the pre-operative CT scan data of the patient with real time intra-operative images acquired using the image acquisition unit during the PCNL surgery, and is used along with an artificial intelligence engine for any or a combination of: identifying kidney and rib position of the patient, predicting an optimal puncture path for the PCNL surgery, and predicting an entry point on the marker patch for the PCNL surgery.

### TECHNICAL FIELD

The present disclosure relates to various systems and devices for renal puncturing. In particular, it pertains to a system for renal puncturing assistance that enables renal puncturing more easily and with reduced risk.

### BACKGROUND

The background description includes information that may be useful in understanding the present invention. It is not an admission that any of the information provided herein is prior art or relevant to the presently claimed invention, or that any publication specifically or implicitly referenced is prior art.

Kidney stones, or renal calculi, are solid masses made of crystals. Kidney stones usually originate in human kidneys. However, they can develop anywhere along your urinary tract, which consists of these parts: kidneys, ureters, bladder and urethra. Kidney stones are one of the most painful medical conditions. The causes of kidney stones vary according to the type of stone.

Percutaneous nephrolithotomy (PCNL) involves removing such stones through a small incision suitably located in a human body (generally in the back), The procedure is resorted to when: the stone causes obstruction and infection or is damaging the kidneys, the stone has grown too large to pass, or pain cannot be controlled.

Percutaneous nephrolithotomy (PCNL) is a primary intervention for removal of kidney stones greater than 2 cm in size or stag horn stones. It is a minimally-invasive procedure to remove stones from kidney by a small puncture wound (up to about 1 cm) through skin, and is most suitable to remove stones of more than 2 cm in size and which are present near pelvic region. It is usually done under general anesthesia or spinal anesthesia.

The access to the defined calyx with correct needle orientation is key to PCNL success. Currently, fluoroscopy and ultrasound (US) are used to image the calyx regions and perform manual puncture to gain access to caliceal system using bull's eye or triangulation techniques. The direction of needle insertion is important for successful access to the predefined calyx. Present systems provide for manual access to the desired calyx is by puncturing the site using free hand technique. An 18 or 21-gauge needle is inserted manually or using an US (ultrasound) needle adaptor to gain access to calyx. In some reports, a needle guiding system fixed to US transducer is reported to guide percutaneous renal access.

However, the manual puncture method as elaborated above is highly dependent on trained and experienced operator and involves steeper learning curve. Prior studies estimate that it takes a minimum of 60 cases to achieve competence in obtaining access and 115 procedures prior to achieving excellence [Tanriverdi et al Eur Urol, 2007. 52(1): p. 206-11., Allen et al, J Endourol, 2005. 19(3): p. 279-82]. Borofsky et al (The Journal of Urology, 195(4, Part 1), 977-981, 2016) has reported that in case of patients which have gone through salvage PCNL treatment, the leading cause (80% cases) of failure in primary treatment was unsuitable access to the stone. Sharma et al has reported that out of 28% terminated surgeries, 38% were due to loss of access, 40% were due to bleeding, and 10% were due to improper angulation during kidney access (Sharma et al, J Dental Med Sci, 2016, 15, 18-25).

Manual renal access involves to and fro movement of the needle for few millimeters as a mean for localization of needle tip. This causes micro traumatization of parenchyma and inadvertent injury to arteries leading to hematoma formation. Manual puncture also involves high cognitive stress for surgeon as imaging provides needle position only in 2D form. It is even challenging when surgeon needs multiple access points for complicated kidney stones.

Further, while using fluoroscopy, surgeons are exposed to harmful radiations. The mean doses to a urologist's eyes, fingers, and collars were measured as 26, 33.5, and 48 μGy per procedure, respectively. The mean effective dose per procedure for an urologist was 12.7 pSv (micro-sievert) (Safak et al, J RadiolProt, 2009, 29: 409-415). Despite the fact that the amount of radiation exposure during the PCNL procedure seems to be minimal while referring to the International Commission on Radiation Protection (ICRP) recommended occupational dose limit of 20 mSv per year, it will be a real concern when the number of PCNL procedures increases with time (Metler et al, Health Phys, 2008, 95: 502-507). Manual renal access causes longer radiation exposure times.

While using ultra sound guidance during manual renal access, close co-ordination is required between one hand of an operator holding ultra sound probe and another holding needle. The needle guiding system attached to US transducer can predict needle position using specialized software. However, angle of entry to caliceal system is limited. While handling manually, in the longitudinal approach, the needle sometimes enters the skin at an oblique angle, which may make subsequent tract dilation and lithotripsy more challenging (Chu et al, 2016, DOI: 10.1089/end.2015.0185). Chau et al achieved renal access in only 83.3% cases using US with navigation system under magnetic field (J Endourology 2016, 30, 160-164).

There is, therefore, a need in the art for a system for renal puncturing assistance that can enable more precise renal puncturing and further guidance of the needle to minimize trauma and aid in quicker stone removal, while also minimizing radiation exposure for the user.

### OBJECT OF THE PRESENT DISCLOSURE

Some of the objects of the present disclosure, which at least one embodiment herein satisfies are as listed herein below.

It is an object of the present disclosure to provide a system for renal puncturing assistance in a percutaneous nephrolithotomy (PCNL) surgery.

It is an object of the present disclosure to provide a system for renal puncturing assistance in a PCNL surgery, which provides real-time augmented reality of renal area to assist a user during the PCNL surgery.

It is an object of the present disclosure to provide a system for renal puncturing assistance during PCNL surgery, which determines and provides an optimum puncture path and entry point for renal puncturing.

It is an object of the present disclosure to provide a system for renal puncturing assistance in a PCNL surgery, which has a robotic device to assist a user during the PCNL surgery.

It is an object of the present disclosure to provide a means to assist in posture maintenance of a patient and positioning the patient in same alignment and position during the PCNL surgery as during the pre-operative CT scan.

### SUMMARY

The present disclosure relates to various systems and devices for renal puncturing. In particular, it pertains to a system for renal puncturing assistance that enables renal puncturing more easily and with reduced risk.

According to an aspect, the present disclosure pertains to a system for renal puncturing assistance in a percutaneous nephrolithotomy (PCNL) surgery, the system comprising: an image processing unit configured to collect a first set of images of a predetermined area corresponding to a renal area of the patient from a pre-operative computed tomography (CT) scan; an image acquisition unit configured to collect a second set of images of the predetermined area of the patient in real-time during the PCNL, surgery; a marker patch provided with one or more markers, the marker patch adapted to be affixed at the predetermined area on the patient, wherein the image processing unit may be configured to monitor the marker patch and generate a first positional data associated with the one or more markers during the pre-operative CT scan, and the image acquisition unit may be configured to monitor the marker patch and generate a second positional data associated with the one or more markers during the PCNL surgery; and a computing unit operatively coupled to the image acquisition unit, the computing unit comprising one or more processors configured to execute one or more instructions stored in a memory of the computing unit and configured to receive the first set of images and the second set of images associated with the predetermined position; overlay the received first set of images on the received second set of images to create an augment reality of the predetermined area and provide real-time position of one or more organs in the renal area of the patient; receive the first positional data and the second positional data associated with the one or more markers; and match the first positional data with the second positional data to provide an operational window for the PCNL surgery; wherein the computing unit may be configured to process the received first set of images and the received second set of images of the predetermined area to identify one or more parameters associated with one or more organs present in the renal area, and determine an optimal puncture path and an entry point for renal puncturing during the PCNL surgery.

In an aspect, the system may comprise a display unit operatively coupled the computing unit, and wherein the display unit may be configured to display augmented reality of any or a combination of the predetermined area of the patient, the optimal puncture path and the entry point for puncturing in the renal area, and the one or more organs present in the renal area, during the PCNL.

In another aspect, the system may comprise a robotic device operatively coupled to the computing unit, and wherein the robotic device may be configured to be operated by a user to assist in the PCNL surgery.

In yet another aspect, the robotic device may comprise an end effect or adapted to rotate 360 degrees around an axis of the robotic device, and wherein the robotic device may be configured to facilitate movement of the end effect or to the predetermined area of the patient.

In an aspect, the end effect or may comprise a needle holding mechanism configured to accommodate at least one needle, and a guide wire mechanism configured to accommodate a plurality of guide wires, and wherein the robotic device may facilitate positioning of the at least one needle at the entry point for puncturing in the renal area and may, enable movement of the at least one needle through the optimum puncture path.

In another aspect, the at least one needle may be a two-part needle having a removable inner part such that removal of the inner part of the two-part needle may form a hollow part within the two-part needle, and wherein the hollow part of the two-part needle may facilitate passing of a corresponding guide wire without bending.

In yet another aspect, the image acquisition unit may be configured to monitor real-time position of the at least one needle and the guide wire during the PCNL surgery, and wherein the computing unit may be configured to provide augmented reality of the positions of the at least one needle and the guide wire to the user during the PCNL surgery.

In an aspect, the system may comprise a PCNL cushion to assist in posture maintenance of the patient and may facilitate positioning the patient in same alignment and position during the PCNL surgery as during the pre-operative CT scan.

In another aspect, the one or more parameters associated with the one or more organs present in the renal area may be any or a combination of a presence of kidney stone, kidney structure, position of kidney and rib, and organs in vicinity of the kidney.

In yet another aspect, the image acquisition unit may comprise any or a combination of at least one camera and at least one fluoroscopic imaging unit, and wherein the image acquisition unit may be coupled to any or a combination of the end effect or and the computing unit.

### BRIEF DESCRIPTION OF DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure. The diagrams are for illustration only, which thus is not a limitation of the present disclosure, and wherein:

FIG. 1 illustrates over all construction of a robotic device (RD) of the proposed system, in accordance with an exemplary embodiment of the present disclosure.

FIGS. 2A to 2C illustrate construction of an end effect or of the proposed system in accordance with an exemplary embodiment of the present disclosure.

FIG. 3 depicts the design of a marker patch in accordance with an exemplary embodiment of the present disclosure.

FIG. 4 depicts design of a PCNL cushion in accordance with an exemplary embodiment of the present disclosure.

FIG. 5 illustrates an overall architecture of the proposed system, in accordance with an exemplary embodiment of the present disclosure.

FIG. 6 illustrates an exemplary augmented reality view displayed by the proposed system during the PGA, surgery, in accordance with an embodiment of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

In the following description, numerous specific details are set forth in order to provide a thorough understanding of embodiments of the present invention. It will be apparent to one skilled in the art that embodiments of the present invention may be practiced without some of these specific details.

Embodiments of the present invention include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, and firmware and/or by human operators.

Embodiments of the present invention may include a computer program product, which may include a machine-readable storage medium tangibly embodying thereon instructions, which may be used to program a computer (or other electronic devices) to perform a process. The machine-readable medium may include, but is not limited to, fixed (hard) drives, magnetic tape, floppy diskettes, optical disks, compact disc read-only memories (CD-ROMs), and magneto-optical disks, semiconductor memories, such as ROMs, PROMs, random access memories (RAMs), programmable read-only memories (PROMs), erasable PROMs (EPROMs), electrically erasable PROMs (EEPROMs), flash memory, magnetic or optical cards, or other type of media/machine-readable medium suitable for storing electronic instructions (e.g., computer programming code, such as software or firmware).

Various methods described herein may be practiced by combining one or more machine-readable storage media containing the code according to the present invention with appropriate standard computer hardware to execute the code contained therein. An apparatus for practicing various embodiments of the present invention may involve one or more computers (or one or more processors within a single computer) and storage systems containing or having network access to computer program(s) coded in accordance with various methods described herein, and the method steps of the invention could be accomplished by modules, routines, subroutines, or subparts of a computer program product.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities or dimensions of items, and so forth, used to describe and claim certain embodiments of the invention are to be understood as being modified in some instances by the term “about”. Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

The recitation of ranges of values herein is merely intended to serve as a shorthand method of referring individually to each separate value falling within the range, Unless otherwise indicated herein, each individual value is incorporated into the specification as if it were individually recited herein.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

Exemplary embodiments will now be described more fully hereinafter with reference to the accompanying drawings, in which exemplary embodiments are shown. These exemplary embodiments are provided only for illustrative purposes and so that this disclosure will be thorough and complete and will fully convey the scope of the invention to those of ordinary skill in the art. The invention disclosed may, however, be embodied in many different forms and should not be construed as limited to the embodiments set forth herein. Various modifications will be readily apparent to persons skilled in the art. The general principles defined herein may be applied to other embodiments and applications without departing from the spirit and scope of the invention. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

Thus, for example, it will be appreciated by those of ordinary skill in the art that the diagrams, schematics, illustrations, and the like represent conceptual views or processes illustrating systems and methods embodying this invention. The functions of the various elements shown in the figures may be provided through the use of dedicated hardware as well as hardware capable of executing associated software. Similarly, any switches shown in the figures are conceptual only. Their function may be carried out through the operation of program logic, through dedicated logic, through the interaction of program control and dedicated logic, or even manually, the particular technique being selectable by the entity implementing this invention. Those of ordinary skill in the art further understand that the exemplary hardware, software, processes, methods, and/or operating systems described herein are for illustrative purposes and, thus, are not intended to be limited to any particular named element.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as a device described herein may be oriented in any desired direction.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims.

Various terms as used herein are shown below. To the extent a term used in a claim is not defined below, it should be given the broadest definition persons in the pertinent art have given that term as reflected in printed publications and issued patents at the time of filing.

Within the scope of this application it is expressly envisaged that the various aspects, embodiments, examples and alternatives set out in the preceding paragraphs, in the claims and/or in the following description and drawings, and in particular the individual features thereof, may be taken independently or in any combination. Features described in connection with one embodiment are applicable to all embodiments, unless such features are incompatible.

Various objects, features, aspects and advantages of the present disclosure will become more apparent from the following detailed description of preferred embodiments, along with the accompanying drawing figures in which like numerals represent like features:

The present disclosure relates to various systems and devices for renal puncturing. In particular, it pertains to a system for renal puncturing assistance that enables renal puncturing more easily and with reduced risk.

In an aspect, the present disclosure pertains to a system for renal puncturing assistance in a percutaneous nephrolithotomy (PCNL) surgery, the system including: an image processing unit configured to collect a first set of images of a predetermined area corresponding to a renal area of the patient from a pre-operative computed tomography (CT) scan; an image acquisition unit configured to collect a second set of images of the predetermined area of the patient in real-time during the PCNL surgery; a marker patch provided with one or more markers, the marker patch adapted to be affixed at the predetermined area on the patient, wherein the image processing unit can be configured to monitor the marker patch and generate a first positional data associated with the one or more markers during the pre-operative CT scan, and the image acquisition unit can be configured to monitor the marker patch and generate a second positional data associated with the one or more markers during the PCNL surgery; a computing unit operatively coupled to the image acquisition unit, the computing unit including one or more processors configured to execute one or more instructions stored in a memory of the computing unit and configured to. receive the first set of images and the second set of images associated with the predetermined position; overlay the received first set of images on the received second set of images to create an augment reality of the predetermined area and provide real-time position of one or more organs in the renal area of the patient; receive the first positional data and the second positional data associated with the one or more markers; and match the first positional data with the second positional data to provide an operational window for the PCNL surgery; wherein the computing unit can be configured to process the received first set of images and the received second set of images of the predetermined area to identify one or more parameters associated with one or more organs present in the renal area, and determine an optimal puncture path and an entry point for renal puncturing during the PCNL surgery.

In an embodiment, the system can include a display unit operatively coupled the computing unit. The display unit can be configured to display augmented reality of any or a combination of the predetermined area of the patient, the optimal puncture path and the entry point for puncturing in the renal area, and the one or more organs present in the renal area, during the PCNL.

In an embodiment, the system can include a robotic device operatively coupled to the computing unit, and wherein the robotic device (also referred to as RD, herein) can be configured to be operated by a user to assist in the PCNL surgery.

In an embodiment, the robotic device can include an end effect or adapted to rotate 360 degrees around an axis of the robotic device. The robotic device can be configured to facilitate movement of the end effect or to the predetermined area of the patient.

In an embodiment, the end effect or can include a needle holding mechanism configured to accommodate at least one needle (also referred to as needle, herein), and a guide wire mechanism configured to accommodate a plurality of guide wires (also, referred to as guide wires, herein). The robotic device can facilitate positioning of the at least one needle at the entry point for puncturing in the renal area and can enable movement of the at least one needle through the optimum puncture path.

In an embodiment, the at least one needle can be a two-part needle having a removable inner part such that removal of the inner part of the two-part needle can form a hollow part within the two-part needle. The hollow part of the two-part needle can facilitate passing of a corresponding guide wire without bending.

In an embodiment, the image acquisition unit can be configured to monitor real-time position of the at least one needle and the guide wire during the PCNL surgery. The computing unit can be configured to provide augmented reality of the positions of the at least one needle and the guide wire to the user during the PCNL surgery.

In an embodiment, the system can include a PCNL cushion to assist in posture maintenance of the patient and can facilitate positioning the patient in same alignment and position during the PCNL surgery as during the pre-operative CT scan.

In an embodiment, the one or more parameters associated with the one or more organs present in the renal area can be any or a combination of a presence of kidney stone, kidney structure, position of kidney and rib, and organs in vicinity of the kidney.

In an embodiment, the image acquisition unit can include any or a combination of at least one camera and at least one fluoroscopic imaging unit. The image acquisition unit can be coupled to any or a combination of the end effect or and the computing unit.

In an embodiment, the one or markers of the marker patch can be radio opaque markers in a pre-determined configuration and can be affixed on the patient to enable the system to store the first positional data of the markers during the pre-operative CT scan, and the first positional data can be matched with the second positional data derived from one or more markers on the patient during real time intra-operative image acquisition while creating the operational window for the PCNL surgery.

In an embodiment, the PCNL cushion can include belt and lock system to ensure that placement and tightness of the cushion onto the patient body remains maintained during the pre-operative CT scan and during the PCNL surgery. The PCNL cushion can facilitate maintaining the position of the renal system during the pre-operative and intra-operative image acquisition assisting the image-processing unit. The PCNL cushion can have a Velcro based design configured to maintain its stability during different surgical positions of the patient.

In an embodiment, the system can include a patient positioning sub-system that can receive inputs from any or a combination of the PCNL cushion and the marker patch to assist the user to position any or a combination of the PCNL cushion and the marker patch with respect to the patient as appropriate.

In an embodiment, the end effect or can be configured to accommodate needles of different sizes and shapes with the corresponding plurality of guide wires.

In an embodiment, movement of the guide wire from an entry point in the robotic device to the needle can be via a channel that prevents bending and/or kinking of the guide wire.

In an embodiment, the needle holding mechanism can create a cavity for movement of the puncture needle according to breathing pattern of the patient.

In an exemplary embodiment, the RD can have six degrees of freedom (56DOF), and can reach one third of the patient's body from all spherical angles.

In an exemplary embodiment, the computing unit can be any or a combination of a Microcontroller, a Microprocessor, and a computer, but not limited to the likes.

In an implementation, the marker patch can be placed on a patient's skin and the specially designed PCNL cushion can be placed around the patient's waist to align the patient in proper position. During intra-operation (that is, during PCNL surgery), the marker patch can guide the RD to identify and navigate three-dimensional (3D) space in operational area, as further elaborated.

In an implementation, the proposed system can use the first set of images gathered from the pre-operative CT scan and live image acquisition unit stream from the patient to acquire the second set of images in real time during the PCNL surgery and create augmented reality (AR) that can provide virtual insight in the patient's body to enable a surgeon take informed decision during renal puncture. Further, any or combination of adjustment of the PCNL cushion, original or perspective corrected first set of image scan be used by the AR engine to match patient alignment and accurate internal organ position between pre-operative CT scan and second set of images (i.e intra operative images) taken by image acquisition unit. The image acquisition unit for the live image stream can be configured in the end effect or. Any or a combination of the image acquisition unit and sensors present in the RD can monitor/track the needle position as it is advanced during renal puncturing. The AR engine can use fluoroscopic imaging known in the art. The end effect or can insert the needle into desired calyx and through the needle guide wire can be guided to secure the puncture site and perform the PCNL procedure.

In an embodiment, the RD of the proposed system can be operated at a safe distance through an appropriately configured computing unit. Thus, exposure of operator/doctor/surgeon/user (the terms being used interchangeably herein) to harmful radiation/ionic exposure can be avoided/reduced.

In an embodiment, the proposed system can be used for quick and reliable renal puncture for PCNL surgery using the AR engine and the AI engine, through the computing unit, the robotic device of the proposed system. The RD can have an end effect or having an image acquisition unit, a needle holding mechanism and a guide wire mechanism. The needle holding mechanism can be configured to accommodate a puncture needle with corresponding guide wire (and likewise, such needles and wires of different shapes and sizes). The needles and the guide wire can be used to access the caliceal system that has stone required to be removed. Besides, the system can incorporate a PCNL cushion and a marker patch configured as further elaborated.

In an exemplary implementation, during the pre-operative stage the marker patch can be stuck onto a patient's skin above waist area and around 11^{th} and 12^{th} rib. Thereafter, the PCNL cushion can be fixed around the patient's waist and the pre-operative computerized tomography (CT) scan recorded.

The marker patch can be removed during time gap of pre-operative CT scan and operation. When removed, the marker patch can leave behind a color mark on the skin so that marker patch can be placed at the same position during operation. In this manner, using the PCNL cushion and the marker patch as elaborated above, the patient can be aligned in exactly the same position during the PCNL surgery and during pre-operative CT scan.

Just before the PCNL surgery, the patient can be positioned on an operating theater (OT) table and, using the PCNL cushion, placed in same position as during pre-operative CT scan. The marker patch can be applied to the patient. Thereafter the robotic device (RD) can be moved using a telescopic handle at its rear and positioned next to the patient. A manual or electric actuating system can lift wheels of the robotic device so that the robotic device rests on floor upon its solid base and thus becomes immoveable. The computing unit, the display unit and the robotic device can be placed at a pre-determined distance (that can be a minimum of 6 feet) away from the robotic device to minimize radiation exposure to the user during fluoroscopic imaging of the puncture site etc.

In an embodiment, the determined optimal puncture path can help the needle held in the end effect or to reach a caliceal system in the renal area of the patient and the desired calyx with minimal trauma to the patient. The user/surgeon can either confirm the optimal puncture path or can update it using his/her experience and judgment. Thereafter, the proposed system can position the needle just above puncture entry point on skin of the patient. If required, the accuracy of the needle position can be confirmed using the real-time second set of images.

In an embodiment, a joystick control can be provided in the proposed system that can in turn be operatively coupled to the computing unit to enable the user of the proposed system advance the needle placed inside the end effect or for puncture. The computing unit can control the RD and its various arms per the user's commands, and can as well enable on the display unit all relevant imaging parameters such as the first set of images during the pre-operative CT scan, the second set of images during the PCNL surgery, the optimal puncture path to follow etc.

In an embodiment, the system can record travel distance of the needle. Once the needle reaches in the caliceal system, inner part of the two-part needle can be removed so as to create a hollow space in the needle for passing the guide wire as known during the PCNL surgery.

Thereafter, the corresponding guide wire can be placed/engaged in the guide wire mechanism provided in the RD and the user can advance the guide wire. Placement of the guide wire in the patient can be observed using the image acquisition unit. The needle with guide wire passing through its hollow part can be disengaged from the end effect or. The RD can next be brought to its idle position.

Thereafter, wheels of the RD can be brought down to touch the floor (by, for instance, releasing hydraulic pressure in a hydraulic system configured to move the wheels up and down). Once the wheels rest on the floor (the RD in turn resting on the wheels), the RD can easily be moved away from the OT table.

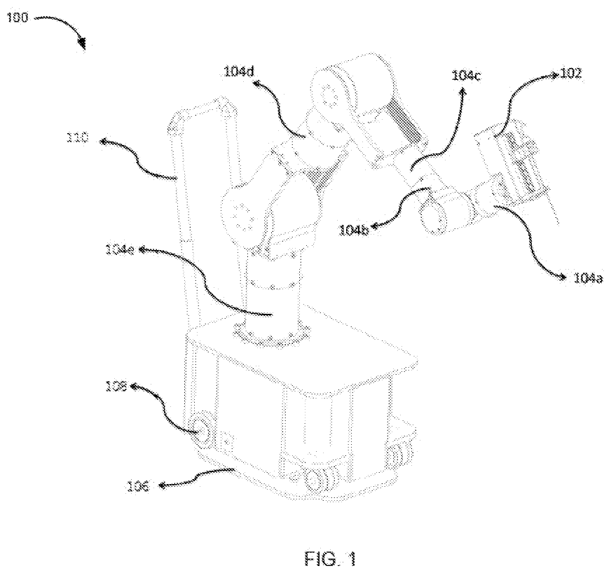

FIG. 1 illustrates over all construction of a robotic device (RD) of the proposed system, in accordance with an exemplary embodiment of the present disclosure.

As illustrated, in an embodiment, a robotic device (RD) 100 of the proposed system can be divided into six main parts viz. an end effect or 102, five arms 104a, 104b, 104c, 104d and 104e, and a base 106.

In an exemplary embodiment, the end effect or 102 can be 150 millimeter (mm) long, 84 mm wide and 230 mm height and can rotate in a 360 degrees circle around an axis with which it is connected to arm 104a. Arms 104 can serve to provide necessary degree of freedom as well as travel as required to the end effect or 102.

In an exemplary embodiment, the arms 104a, 104b, 104c, 104d, and 104e can be 180 mm, 150 mm, 300 mm, 550 mm, and 350 mm long respectively. Arm 104a can have a side joint, arm 104b can have a revolute joint, while arm 104e can have a base joint. Both these joints can rotate in 360 degrees. Arm 104c and arm 104d can rotate at angles of 260 degrees and 130 degrees respectively.

In an embodiment, the base 106 can include an actuation mechanism to raise it up so that the RD 100 rests on wheels 108 when using telescopic handle 110 the RD can be easily transported. The actuation mechanism can as well be used to lower the base 106 and rest it on the floor so as to stabilize the RD during an operation.

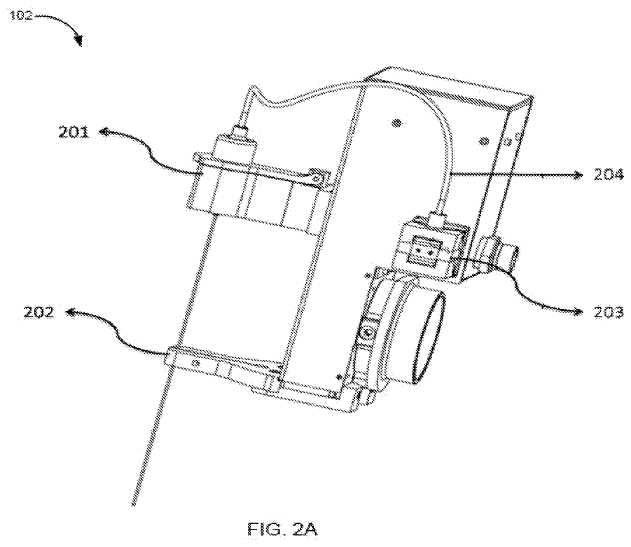

FIGS. 2A to 2C illustrate construction of an end effect or of the proposed system in accordance with an exemplary embodiment of the present disclosure.

As illustrated in FIG. 2A, the end effect or 102 can has a needle holding mechanism. The needle holding mechanism can have a needle holding mechanism cap 208 that can include an upper needle holding block 202a at its lower surface, and a lower needle holding block 202b as shown. The needle holding mechanism can hold PCNL needles of varying lengths and shapes. In an exemplary embodiment, the needles can vary in length from 7 centimeter (cm) to 20 cm, but not limited to the likes.

In an embodiment, the end effect or 102 can have a guide wire entry assembly 204 that can push a guide wire through the needle held in needle holding mechanism. In an exemplary embodiment, the guide wires can have diameter of 0.53 millimeter (mm) to 0.64 mm, but not limited to the likes. The lower needle holding block 202b of the needle holding mechanism can be transparent with low auto fluorescence and can have a guiding slot to maintain axis of puncture without any deflection of needle held in needle holding mechanism.

In an embodiment, the end effect or 102 can have limit sensors at its both ends so as to limit movement of the needle held within. Further, it can have at least one image acquisition unit to capture and record images during surgery for image processing.

In an embodiment, a tube 206 can enable transfer of the guide wire without bending and kinking from guide wire entry assembly 204 to needle holding mechanism cap 208 further illustrated in FIG. 2B.

As illustrated in FIG. 2B, in an embodiment, the needle holding mechanism cap 208 can include to conical two conical structures 222 and 224 to direct the guide wire into hollow part of the needle held in upper needle holding block 202a. The movable cone 224 can slide over fixed cone 222 when the needle has two parts. As known, the needle used for the PCNL procedure can include two parts with an external hollow part enclosing a central part. The central part can be removed when required to create a hollow space/tube through Which the guide wire can be passed.

In an embodiment, when central part of the needle is removed, the movable cone 224 can come down closer to opening of the hollow outer part of the needle thereby creating a channel for guide wire to move into hollow part of the needle without bending.

In an embodiment, the needle holding mechanism can include a needle casing 226, which can have a round outer surface fitting into needle holding mechanism. Inner part of the needle casing can have a mold that can hold the outer surface of the needle cap 208 of varying shapes. Guide wire entry assembly 204 can have guiding slots (242, 244, as shown in FIG. 2C) to ensure entry of the guide wire without bending. A motorized system can run two rollers shown as 246 in FIG. 2C that can move the guide wire into tube 206 (FIG. 2A) that is connected to needle holding mechanism cap 208 (FIG. 2A). In this manner, the guide wire inserted into slot 242 can be guided into a hollow needle held in end effect or 102.

In an embodiment, the needle holding mechanism can create a cavity when required for needle movement according to the breathing pattern of patient during puncture or post puncture. Appropriate parts of the needle holding mechanism 210 (for instance the upper and lower needle holding blocks 202a and 202b respectively) can be made up of clear, transparent, low auto fluorescence material.

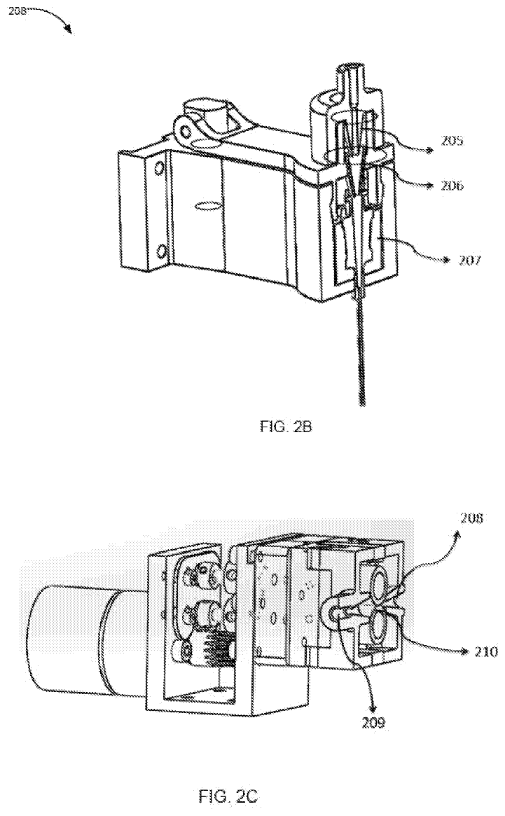

FIG. 3 depicts the design of a marker patch in accordance with an exemplary embodiment of the present disclosure.

In an exemplary embodiment, a marker patch 300 of the proposed system can have a marker patch window 302 (also referred to as operational window 302 or window 302, herein) of dimensions 100 mm×100 mm (length×width) as shown, but not limited to the likes. The marker patch window 302 can include one or more markers. In an exemplary embodiment, the one or more markers can include four CT opaque markers 304 (collectively referred to as opaque markers 304, herein) at four corners or edges of the window 302. Two parallel edges of the window 302 can have stick arms (306) to stick the marker patch 300 on a patient's skin. The opaque markers 304 can have a sign (such as a plus or a cross sign printed on them). Such signs can be recognized during image processing. In an exemplary embodiment, stick arms 306 can be 100 mm long as shown.

In an embodiment, the marker patch 300 can be affixed on a patient during a pre-operative CT scan when proposed system can store the first positional data of the markers 304. During real time intra-operative image acquisition proposed system can match the first positional data with that derived from markers 304 on the patient so as to ensure that the patient is in same position as he/she was during the pre-operative scan. The operational window 302 on the marker patch can be configured as an object surface sterilization window.

In an embodiment, the marker patch 300 can be used to assist creation and mapping of augmented reality (AR) using image processing via an augmented reality (AR) engine. The process can include augmenting pre-operative CT scan data of a patient with real time intra-operative images acquired using image acquisition unit in the end effect or, and the AR engine can be used along with an artificial intelligence (AI) engine for any or a combination of: identifying kidney and rib position of the patient, predicting an optimal puncture path for the surgery, and predicting an entry point on the marker patch for the PCNL surgery.

In an embodiment, the image acquisition unit on the end effect or can record patient images. Markers on the patient body can be registered in the proposed system and aligned with markers registered in the CT scan data. Further, position of internal organs can be mapped on AR image.

In an exemplary embodiment, if required, three-dimensional volume of CT scan data can be corrected/calibrated. Proposed system can enable determination of puncture site pre-operatively or after such calibration.

In an embodiment, the AR created using first set of images from the pre-operative CT scan and the second set of images during the PCNL surgery from the image acquisition unit on the end effect or can be displayed on a display of the display unit enabled by the computing unit of the proposed system.

It is to be appreciated that the marker patch 300 are taken out post pre-operative CT scan and reattached just before the PCNL surgery. However, proposed system imposes no such requirement and the marker patch 300 can remain stuck to the patient's body. The marker patch 300 can maintain distance between the markers 304 permanently, and can leave behind positional marks when removed for easy re-attachment if required to the patient.

In an embodiment, the, marker patch proposed can provide a user/surgeon with sufficient area to allow the required disinfection/sterilization of the skin surface before puncture and during surgery.

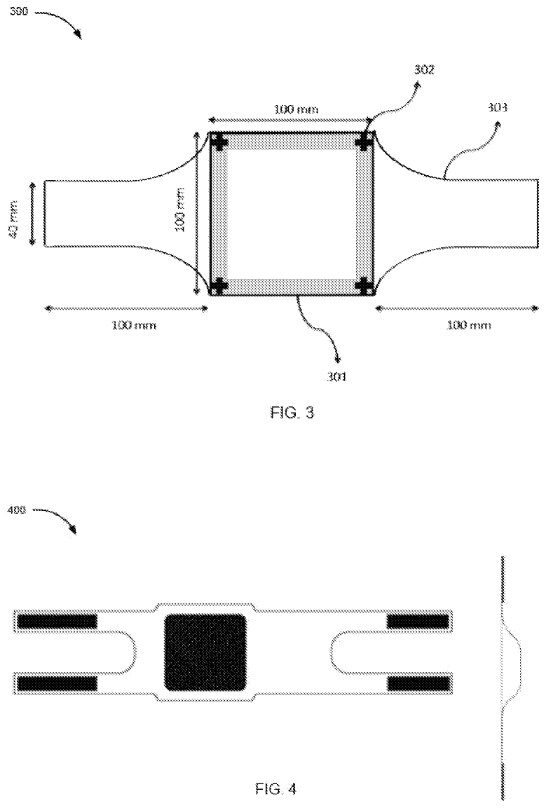

FIG. 4 depicts design of a PCNL cushion in accordance with an exemplary embodiment of the present disclosure.

Anatomically, kidneys are held to their positions by soft tissues. The flexibility of these soft tissues provides mobility (though limited) to kidneys. Such mobility can be caused by the patient posture, inspiration, expiration, or any external force. This mobility can be as high as −1 cm, which can severely jeopardize the accuracy of the internal organ position estimation. The PCNL cushion 400 of the proposed system can arrest the mobility of the kidneys. The PCNL, cushion 400 can be affixed onto the patient around his/her waist, which compresses the free space in renal area to constraint the motion of the kidneys. The PCNL cushion 400 can restrict the motion of the kidney to few mm to provide accurate renal puncturing operation.

In an exemplary embodiment, the PCNL cushion 400 can include a central cushion area 402 of dimensions 28 centimeters (cm)×28 cm×10 cm (length×width×thickness) connected to an appropriate fastening mechanism such as Velcro straps 404 and 406 illustrated. Straps 404 and 406 can be attached to two parallel edges of central cushion area 402.

In an embodiment, the cushion straps can have a combination Velcro and belt system to tighten the belt around the patient's waist to maintain the PCNL cushion tightening and position accuracy during CT scan and during PCNL procedure (intra-operative phase). A sticky skin marker can be used to record the position of the PCNL cushion during on a patient CT scan phase and then repeat the same position during the operation phase, to ensure correct positioning of the patient.

In an embodiment, the PCNL cushion 400 can include a belt and lock system to ensure that the placement and tightness of the cushion onto the patient body remains maintained before and during puncture. Further, Velcro straps 404 and 406 can maintain the stability of the cushion during different surgical positions such as prone or supine positions.

In an exemplary embodiment, to ensure that the alignment of internal body organs is same with use of PCNL cushion, the second set of images during the PCNL surgery can be taken at more than one angle and compare with AR generated by AR engine.

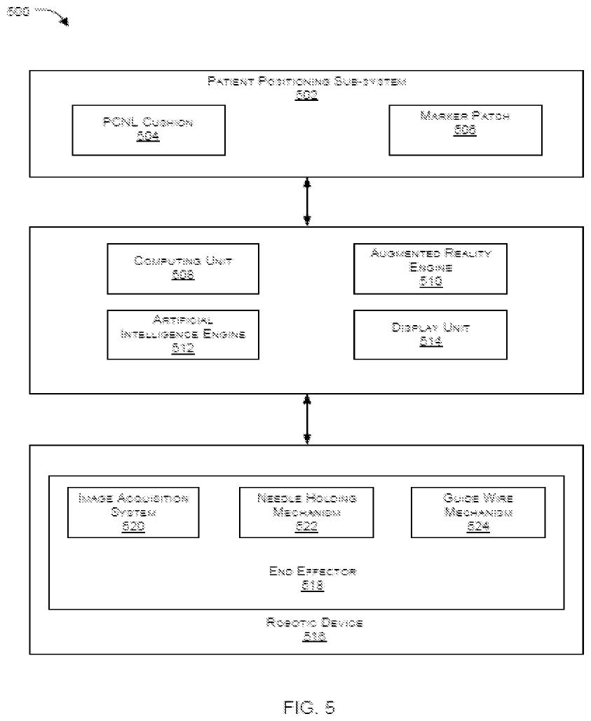

FIG. 5 illustrates an overall architecture of the proposed system, in accordance with an exemplary embodiment of the present disclosure.

As illustrated, in an embodiment, the proposed system for renal puncturing assistance can include a patient position sub-system 502, which can include a marker patch 504 and a PCNL cushion 506 to assist in posture maintenance of the patient and facilitates positioning the patient in same alignment and position during the PCNL surgery as during the pre-operative CT scan. The system can further include a computing unit 508, an augmented reality engine 510, an artificial intelligence engine 512, a display unit 514, and a robotic device 514. The robotic device 514 can include an end effect or 516. End effect or 516 can have an image acquisition unit, a needle holding mechanism, and a guide wire mechanism.

In an embodiment, the PCNL cushion 504 and the marker patch 506 can provide necessary inputs to patient positioning sub-system 502 to enable the patient to be kept in same position during pre-operative CT scan as well as during PCNL surgery as described. Further, computing unit 508 can receive inputs from various components and patient positioning sub-system 502 and can in turn enable a user control the RD 516, the end effect or 518 etc. as already elaborated.

As described, PCNL cushion 504 can assist maintain correct posture of the patient during the pre-operative computerized tomography (CT) scan and during the PCNL surgery, and can assist position the patient in same alignment and position with respect to the PCNL cushion during the PCNL surgery as during the CT scan. For the purpose, the PCNL cushion 504 can be configured to provide necessary inputs to patient positioning sub-system 502 and such inputs can be displayed on a display unit 514 controlled by the computing unit 508 to help the user of proposed system position the PCNL cushion with respect to the patient as appropriate.

In an embodiment, both audio as well as video signals can be provided to the user to assist the user to position the PCNL cushion 504 as appropriate. For instance. PCNL cushion 504 may have RFID devices/markers or pressure sensors or both transmitting their position to sub-system 504 for the purpose.

It is to be appreciated that any means that can assist position the patient in same alignment and position with respect to the PCNL cushion during the PCNL surgery as during the CT scan are fully a part of the present disclosure.

In an embodiment, the marker patch 506 can assist creation and mapping of augmented reality (AR) using image acquisition unit 520 via the AR engine 510.

In an embodiment, the robotic device 516 can be configured to be operated via the computing unit 512 using computer vision assisted by the AR engine 510. The AR engine 510 can augment the first set of images during the pre-operative CT scan of the patient with real time second set of images acquired using the image acquisition unit 520 during the PCNL surgery. Further, the AR engine 510 can be used along with the AI engine 512 to provide any or a combination of; identifying kidney and rib position of the patient, predicting an optimal puncture path for the PCNL surgery, and predicting an entry point on the marker patch for the PCNL surgery. The RD 516 can as well operate puncture needle held in needle holding mechanism (along the optimal puncture path) and provide a guide wire through the needle as required, under instructions from the user of the proposed system via microcontroller 512.

In an embodiment, the marker patch 506 can carry radio opaque markers in a pre-determined configuration and can be affixed on the patient to enable the system store first positional data of the markers during the pre-operative CT scan. The first positional data can be matched with the second positional data derived from the markers on the patient during real time intra-operative image acquisition while creating an operational window for the PCNL surgery. For the purpose, marker patch 506 can be configured provide necessary inputs to patient positioning sub-system 501 and such inputs can be displayed on the display unit 514 controlled by the computing unit 508 to help the user position the marker patch 504 with respect to the patient as appropriate.

In an embodiment, the computing unit 508 can communicate with one/more motors in the RD 516. The computing unit 508 can receive positional data pertaining to the needle/guide wire by means of the image acquisition unit 520 mounted in the end effect or 516, overlay the first set of images during the pre-operative CT scan data on real time second set of images of the patient being acquired during the PCNL, surgery to create augmented reality and provide real time organ position of the patient on the display unit 514. A user/surgeon can look at the display unit 514 and control the motors using the computing unit 508 to actuate as appropriate movements of arms of the RD 516, the needle (held in end effect or 518) and the guide wire (that is inserted through guide wire entry assembly shown as 204 in FIG. 2).

In this manner, as elaborated above, proposed system augments pre-operative CT scan data with real time intra-operative images acquired while the PCNL procedure is underway using image acquisition unit mounted on the end effect or and processes such data using an AR engine. This creates augmented reality that can be displayed on a display operatively connected to the computing unit of the proposed system. (or can be as well a part of the system). Further, artificial intelligence (AI) engine of the proposed system can use pre-operative CT scan data to identify or register kidney stone(s), kidney structure, other organs in the vicinity, kidney and rib positions of the patient in a corrected three dimensional volume, and position of the markers on the skin to predict an optimal puncture path and entry point for the needle on the patch. Accordingly, information can be sent to the RD to position the needle at puncture entry point. The augmented reality (AR) engine can enable display of CT scan data/corrected three-dimensional volume of the CT scan data, real time patient images, predicted puncture path, and needle entry point. The needle entry point and travel positional information can be used by proposed system to continuously move the needle along the optimal puncture path. Using the microcontroller, arms of the RD can be moved to position the needle according to the target. The AR engine can display the virtual path created on a display system. A surgeon/user can control the microcontroller to accordingly operate the RD to puncture the kidney and gain access to the stone(s).

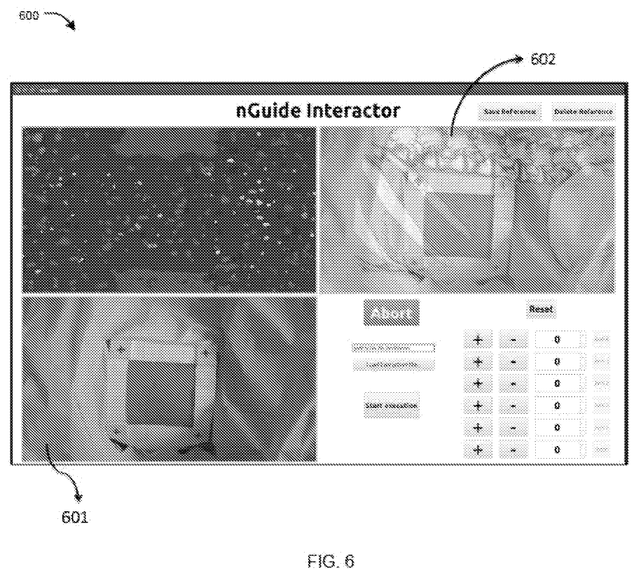

FIG. 6 illustrates an exemplary augmented reality view displayed by the proposed system during the PCNL surgery, in accordance with an embodiment of the present disclosure.

In an embodiment, the system can receive the CT scan data from the image processing unit. It can then be passed on to the AI module of the system for renal organ detection, bone detection (ribs), and marker detection. In the meanwhile, the image acquisition unit can detect the position of the markers in the three-dimensional space (as shown in 601). The real-time position of the markers and their positional information from the pre-operative data can be then matched, by changing the scale and orientation of the rendered volume obtained from pre-operative CT scan, to the marker positional information from the image acquisition unit. The transparencies of both views can be adjusted and views can be overlaid onto each other (as shown in 602). This overlay can generate the augmented reality view (602) for the user on the display unit. The AI module can further analyze the pre-operative data and corrects it after obtaining the fluoroscopy data from the image acquisition unit. The corrected data can represent intra-operative internal organ positions. This view can be further analyzed to obtain the optimal puncture path and point of skin to puncture. Then, the line of puncture can be programmatically, generated on the augmented reality view.

Although the proposed system has been elaborated as above to include all the main components, it is completely possible that actual implementations may include only a part of the proposed components or a combination of those or a division of those into sub-components in various combinations across multiple devices that can be operatively coupled with each other, including in the cloud. Further the components can be configured in any sequence to achieve objectives elaborated. Also, it can be appreciated that proposed system can have, or be operatively connected to a computing device or across a plurality of computing devices operatively connected with each other, wherein the computing devices can be any of a computer, a laptop, a smartphone, an Internet enabled mobile device and the like. Therefore, all possible modifications, implementations and embodiments of where and how the proposed system is configured are well within the scope of the present invention.

As used herein, and unless the context dictates otherwise, the term “coupled to” is intended to include both direct coupling (in which two elements that are coupled to each other or in contact with each other) and indirect coupling (in which at least one additional element is located between the two elements). Therefore, the terms “coupled to” and “coupled with” are used synonymously. Within the context of this document terms “coupled to” and “coupled with” are also used euphemistically to mean “communicatively coupled with” over a network, where two or more devices are able to exchange data with each other over the network, possibly via one or more intermediary device.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

While some embodiments of the present disclosure have been illustrated and described, those are completely exemplary in nature. The disclosure is not limited to the embodiments as elaborated herein only and it would be apparent to those skilled in the art that numerous modifications besides those already described are possible without departing from the inventive concepts herein. All such modifications, changes, variations, substitutions, and equivalents are completely within the scope of the present disclosure. The inventive subject matter, therefore, is not to be restricted except in the spirit of the appended claims.

### ADVANTAGES OF THE INVENTION

The proposed disclosure provides a system for renal puncturing assistance in a percutaneous nephrolithotomy (PCNL) surgery.

The proposed disclosure provides a system for renal puncturing assistance in a PCNL surgery, which provides real-time augmented reality of renal area to assist a user during the PCNL surgery.

The proposed disclosure provides a system for renal puncturing assistance during PCNL surgery, which determines and provides an optimum puncture path and entry point for renal puncturing.

The proposed disclosure provides a system for renal puncturing assistance in a PCNL surgery, which has a robotic device to assist a user during the PCNL surgery.

The proposed disclosure provides a means to assist in posture maintenance of a patient and positioning the patient in same alignment and position during the PCNL surgery as during the pre-operative CT scan.

We claim:

1. A system for renal puncturing assistance in a percutaneous nephrolithotomy (PCNL) surgery, the system comprising:
an image processing unit configured to collect a first set of images of a predetermined area corresponding to a renal area of the patient during a pre-operative computed tomography (CT) scan;
an image acquisition unit configured to collect a second set of images of the predetermined area of the patient in real-time during the PCNL surgery;
a marker patch provided with one or more markers, the marker patch adapted to be affixed at the predetermined area on the patient, wherein the image processing unit is configured to monitor the marker patch and generate a first positional data associated with the one or more markers from the pre-operative CT scan, and the image acquisition unit is configured to monitor the marker patch and generate and a second positional data associated with the one or more markers during the PCNL surgery; and
a computing unit operatively coupled to the image acquisition unit, the computing unit comprising one or more processors configured to execute one or more instructions stored in a memory of the computing unit and configured to:
receive the first set of images and the second set of images associated with the predetermined position;
overlay the received first set of images on the received second set of images to create an augment reality of the predetermined area and provide real-time position of one or more organs in the renal area of the patient;
receive the first positional data and the second positional data associated with the one or more markers; and
match the first positional data with the second positional data to provide an operational window for the PCNL surgery;
wherein the computing unit is configured to process the received first set of images and the received second set of images of the predetermined area to identify one or more parameters associated with one or more organs present in the renal area, and determine an optimal puncture path and an entry point for renal puncturing during the PCNL surgery.

2. The system as claimed in claim 1, wherein the system comprises a display unit operatively coupled the computing unit, and wherein the display unit is configured to display augmented reality of any or a combination of the predetermined area of the patient, the optimal puncture path and the entry point for puncturing in the renal area, and the one or more organs present in the renal area, during the PCNL.

3. The system as claimed in claim 1, wherein the system comprises a robotic device operatively coupled to the computing unit, and wherein the robotic device is configured to be operated by a user to assist in the PCNL surgery.

4. The system as claimed in claim 3, wherein the robotic device comprises an end effect or adapted to rotate 360 degrees around an axis of the robotic device, and wherein the robotic device is configured to facilitate movement of the end effect or to the predetermined area of the patient.

5. The system as claimed in claim 4, wherein the end effect or comprises a needle holding mechanism configured to accommodate at least one needle, and a guide wire mechanism configured to accommodate a plurality of guide wire, and wherein the robotic device facilitates positioning of the at least one needle at the entry point for puncturing in the renal area and enables movement of the at least one needle through the optimum puncture path.

6. The system as claimed in claim 5, wherein the at least one needle is a two-part needle having a removable inner part such that removal of the inner part of the two-part needle forms a hollow part within the two-part needle, and wherein the hollow part of the two-part needle facilitates passing of a corresponding guide wire without bending.

7. The system as claimed in claim 5, wherein the image acquisition unit is configured to monitor real-time position of the at least one needle and the guide wire during the PCNL surgery, and wherein the computing unit is configured to provide augmented reality of the positions of the at least one needle and the guide wire to the user during the PCNL surgery.

8. The system as claimed in claim 1, wherein the system comprises PCNL cushion to assist in posture maintenance of the patient and facilitates positioning the patient in same alignment and position during the PCNL surgery as during the pre-operative CT scan.

9. The system as claimed in claim 1, wherein the one or more parameters associated with the one or more organs present in the renal area are any or a combination of a presence of kidney stone, kidney structure, position of kidney and rib, and organs in vicinity of the kidney.

10. The system as claimed in claim 1, wherein the image acquisition unit comprises any or a combination of at least one camera and a fluoroscopic imaging unit, and wherein the image acquisition unit is coupled to any or a combination of the end effect or and the computing unit.
